(19) United States
(12) Patent Application Publication
(10) Pub. No.: US 2026/0210929 A1
(43) Pub. Date: Jul. 23, 2026

(54) METHOD TO CALCULATE THE RISK OF MYCOTOXINS IN POULTRY AND SWINE USING CONTAMINATION DATA AND RISK FACTORS AND ADJUSTMENTS

(71) Applicant: Ricardo Hummes Rauber, Curitiba (BR)

(72) Inventor: Ricardo Hummes Rauber, Curitiba (BR)

(21) Appl. No.: 19/416,427

(22) Filed: Dec. 11, 2025

(30) Foreign Application Priority Data
Jan. 23, 2025 (BR) 102025001371

(51) Int. Cl. G01N 33/02

(52) U.S. Cl. CPC G01N 33/02; G01N 2333/37

(57) ABSTRACT

Method to calculate and verify the risk of mycotoxins for poultry and/or swine, providing information to livestock producers for risk follow-up and mitigation. An evaluation period is created for feeds and feed raw materials for the poultry and/or swine, collection of data related to mycotoxin contamination and, then, calculating the index of risk of contamination by mycotoxins using contamination gross data and their prevalence, in addition to using the epidemiological factor and risk adjustment according to the species, age and animal category analyzed and to create a standardized numerical base among all the mycotoxins analyzed. The method may be used to analyze and calculate the risk of mycotoxin contamination for different species of animals and, more specifically, for the referred risk calculation for poultry and/or swine populations.

### FIELD OF INVENTION

The invention in this patent application discloses a means to calculate and verify the risk of mycotoxins for poultry and/or swine, providing information to livestock producers for risk follow-up and mitigation. The method is conducted by creating an evaluation period for feeds and feed raw materials for the referred poultry and/or swine, collection of data related to mycotoxin contamination and, then, calculating the index of risk of contamination by such mycotoxins using such contamination gross data and their prevalence, in addition to using the epidemiological factor and risk adjustment according to the species, age and animal category analyzed and to create a standardized numerical base among all the mycotoxins analyzed. The method specifically shown herein is specifically used to analyze and calculate the risk of mycotoxin contamination in feeds and feed raw materials for different species of animals and, more specifically, for the referred risk calculation for poultry and/or swine populations.

### Invention Foundations

In the specific livestock area, mycotoxins, toxic substances produced by fungi, cause significant damages to poultry and/or swine digestive tract, immune and reproductive systems, this being, thus, a challenge to be resolved by livestock producers so as to minimize losses related to the zootechnical performance of such animals and, consequently, financial losses to the produces. The long-term exposure, even at low mycotoxin levels, may cause chronic intestine inflammation and immunosuppression, increasing the susceptibility to secondary infections, such as E. coli and Salmonella, of such animals. This justifies the increased impact of prevalence in the risk calculation, because continuous contamination, even at low concentrations, may have significant cumulative deleterious effects on such animals.

Therefore, epidemiological veterinary models frequently use parameters adjusted to reflect the extended and cumulative impact of exposures on poultry and/or swine. The literature on epidemiological modeling, as described by Chubb and Jacobsen, emphasizes that mathematical models can be used to identify sensitive variables and predict health endpoints in animal populations. The sensitivity analysis is crucial to determine how different components of the model influence the final result. High sensitivity parameters, such as prevalence, must be precisely measured and may justify the use of higher powers to reflect their disproportionate impact.

Concerning behavioral models in epidemiology, a study on behavioral epidemic models emphasizes the importance of adjusting parameters to reflect the behavioral response of the population to the perceived risk. In models on which the risk perception changes behaviors (for example, increased adherence to non-pharmacological interventions), adjustment are made to represent how such changes affect the disease dynamics. Similarly, in the context of mycotoxins, the extended prevalence and continuous perception of risk justify the use or higher powers to capture the cumulative impact of exposure.

In its turn, concerning the impact of mycotoxins on poultry and swine health, a number of studies show that the continuous exposure and high prevalence of mycotoxins, such as aflatoxins and fumonisins, have serious and cumulative effects on such poultry and swine health. Such effects include changes to weight gain, food conversion and biochemical parameters, in addition to histopathological damages to vital organs such as the liver, intestine and kidneys.

Thus, this invention application is intended to disclose a method to calculate and verify the risk of mycotoxins to poultry and/or swine, so as to allow individuals interested in such data, such as livestock producers who specifically raise such poultry and/or swine, to have a simple and efficient form of risk analysis so that, if necessary, they take the due health measures given the data obtained related to mycotoxin contamination.

### STATE-OF-THE-ART

Some more relevant state-of-the-art documents were indicates in the search so as to outline the state-of-the-art relevant to the invention. One of such documents is patent US2022074900, entitled “Monitoring mycotoxins and its metabolites in the blood of pigs or broiler chickens” and published on Mar. 10, 2022, the abstract of which being shown below: “This invention refers to a multiple screening method to detect a great number of mycotoxins and metabolites in broiler chicken and swine, the method comprising the collection of broiler chicken and swine blood as a dry blood sample, the preparation of the dry blood sample for analysis and the analysis of the dry blood sample prepared by using a two-step liquid chromatography-tandem mass spectrometry (LC-MS/MS) In a first LC-MS/MS step, for a given mobile phase of the LC process, the mass spectrometer works in the negative electrospray ionization mode and, for the other mobile phase of the LC process, the mass spectrometer operates in the positive electrospray ionization mode. This method can be advantageously used in the screening and evaluation of swine or broiler chicken exposure to feeds contaminated with mycotoxins. In addition, this method can be used to evaluate the impact of adding mycotoxin detoxifying agents to animal feed.”

Another document found is patent KR102152310, entitled “Multi-residue and multi-class analytical method for agricultural chemical veterinary drug and mycotoxin in feed” and published on Sep. 4, 2020, the abstract of which being shown below: “This invention refers to a simultaneous analysis method for pesticide residues in feed, veterinary drugs and mycotoxins and provides a simultaneous analysis method for each feed samples using methods such as GC-MS/MS and LC-MS/MS with only two pre-treatments using the difference of polarity of each material remaining in the feed. Through this invention, it is possible to construct an efficient analysis method which may save time, cost, etc. to monitor harmful substances in the feed, providing an easier analysis method compared with the existing technology which uses approximately 40 methods.”

Another document found is patent US2011306508, entitled “Method of Mycotoxin Detection” and published on Dec. 15, 2011, the abstract of which is shown below: “The presence of mycotoxins in agricultural products requires large scale tests of a broad variety of sample materials to guarantee the safety of food and feeds. Mycotoxin ochratoxin A represents a qualification for all mycotoxins, because the sensitivity level required for such compound regulatory requirements at the parts per billion level is so low or lower than any other mycotoxin. This invention describes the identification of a set of DNA ligands with binding affinity and sufficiently high specificity for ochratoxin A to allow an improvement compared to the existing separation, concentration and quantitative determination methods for ochratocin A in the sample material.”

### Objectives and Advantages of the Invention

The invention disclosed in this invention patent application shows the following specific advantages compared with the state-of-the-art:
The possibility of using different risk evaluation risks in the disclosed calculation allows the evaluation of such risk both in the short- and long-term, both being important for livestock producers in different breeding centers or periods of time to be analyzed;
The use of the epidemiological fact and risk adjustment for each species, age and poultry or swine category allows the adequacy of the calculation for different livestock producers having, thus, flexibility unforeseen in the state-of-the-art; and
In normal analyzes, the verification of information by experts is required given that different mycotoxins present different concentrations in samples, while in the invention of this patent application the risk index is adjusted to a standardized numerical base among all the mycotoxins analyzed, allowing easy verification of the risk of contamination by mycotoxins even by a normal livestock producer.

Thus, the invention shows usability that is not demonstrated in the state-of-the-art, bringing advantages for use by a high range of livestock producers who raise poultry and swine for their appropriate raising and supply to consumers of products obtained from such animals, this being something that is not shown in the state-of-the-art.

### General Description of the Invention

The innovative invention disclosed in this invention patent application shows an evaluation method and risk analysis for existing mycotoxins in feeds and feed raw materials for production animals and, more specifically in poultry and swine raising, such risk detection method being adaptable for livestock producers of different scales for taking management decisions of their respective businesses.

Thus, the method disclosed herein establishes a risk evaluation period of the animals for mycotoxins, the contamination data collection of such mycotoxins in feeds and feed raw materials for relevant and analyzed animals and, then, performing the appropriate risk calculation of mycotoxins using the data obtained from contamination and factors analyzed by the livestock producer, of contamination prevalence in addition to an epidemiological factor and risk adjustment, thus adapting such calculation conducted to a number of possible livestock means of poultry and swine, including, but not restricted to breeding, replacement animals, poultry producers of eggs for consumption and animals intended for slaughter, given the high adaptability of such method.

Thus, this method adapts to a wide range of livestock producers in its calculations conducted for the mycotoxin risk calculation in poultries and swine, this being something not predicted in the verified state-or-the-art.

### DESCRIPTION OF THE FIGURES

The invention will be described below in details being that, for better understanding, references will be made to the attached drawings, on which they are represented:

FIG. 1: Flowchart showing all the main and relevant steps to perform the method.

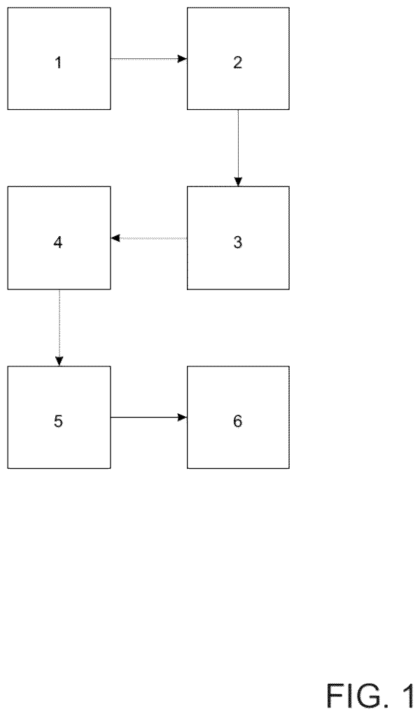

FIG. 2: Graphs showing concentrations in micrograms per kilogram, or parts per billion, of different mycotoxins in different samples;

FIG. 3: Graphs showing adjusted risk index obtained following analysis of different mycotoxins as shown in this method and showing different toxins with risk index with the same standardized numerical base.

The patent or application file contains at least one drawing executed in color. Copies of this patent or patent application publication with color drawing(s) will be provided by the Office upon request and payment of the necessary fee.

### DETAILED DESCRIPTION OF THE INVENTION

The METHOD TO CALCULATE THE RISK OF MYCOTOXINS IN POULTRY AND SWINE USING CONTAMINATION DATA AND RISK FACTORS AND ADJUSTMENTS shows a method that collects mycotoxin contamination data, i.e., several toxic substances produced by different fungi species, in feeds and feed raw materials for production animals and, more specifically, poultry and swine used for human consumption of their meat or secondary products, such as, for example, eggs or milk. The risk is a numerical indicator, or index, showing the risk intensity related to the mycotoxin challenge for animals from the herd of a certain company, producer or region.

This invention shown herein is implemented by means of a method, such method conducted and shown in a series of sequential steps, such steps shown below in this detailed description.

In an initial Step 1 (1) a period of time for data collection to evaluate the risk of contamination is established, such as, for example, per shift, daily, weekly, fortnightly or monthly, the quantity of samples collected in such selected period of time, and also what mycotoxins will be analyzed and followed-up will be selected, those being chosen according to the needs of the user or livestock producer who use such method shown herein. In an implementation of the method, for example, in weekly period with fourteen samples, two sample collections are conducted every day. Such choice of period of time and quantity of sample is to be decided by the user of such method and depending on the user's livestock status, thus, being appropriate to a number of livestock producers. Both the choice of the period of time and the choice of the quantity of samples are already performed by the companies and producers, only providing information concerning the collection of inputs for analysis and performance of the calculations themselves. The choice of the sample collection means, such as, for example, if this is conducted by manual or automatic means, is also decided by the method user according to his/her needs. We noted that the shorter the period of time chosen, higher is the accuracy of the risk calculation performed, this further depending on the number of samples analyzed in such period.

In a Step 2 (2), the collection of samples for analysis of each mycotoxin selected during the period of time chosen is conducted, for then, perform their analyzes. Such collection means uses conventional means to collect data of mycotoxins existing in the state-of-the-art, such as, for example, by using mass spectrometry, immunoenzimatic, infrared assays or any other method available in the market of mycotoxin analyzes in feeds and feed raw materials for production animals. Some means to specifically perform sample collections in the period specified in Step 1 (1) and the analysis itself of mycotoxins are shown in articles “Comparison of the Efficiency between Two Sampling Plans for Aflatoxins Analysis in Maize” [1] and “Dois Planos de Amostragem Para Análise de Fumonisinas Em Milho” [2].

In a Step 3 (3), the average contamination of mycotoxins in micrograms per kilogram, i.e., μg/kg, also known as parts per billion is calculated, of each of the collected samples. The prevalence is also calculates, this meaning a percentage of positive samples in the selected period of time. Such contamination and prevalence calculations are conducted for each mycotoxin selected to be analyzed. The prevalence calculation is thus conducted to obtain the prevalence percentage:

$$Prevalence = positive samples / total of samples \times 100$$

In a Step 4 (4), the choice of an epidemiological factor, or FE, of between 1.1 and 1.9 must be conducted, being that it must be applied as a calculation factor in the prevalence calculated in Step 3 (3), and such factor ranging according to different cases of use. The choice of the epidemiological factor is conducted by an expert in mycotoxins in production animals according to the needs of a specific livestock situation, because it involves the knowledge of such toxins' mechanisms of action in the animals' body, as well as the impact of continuous or punctual exposures.

In a Step 5 (5), a calculation of the mycotoxin risk index is performed, then, it should be applied to each mycotoxin analyzed by the method, with the main performance using the following equation:

$$RM = Contamination \times {Prevalence}^{FE}$$

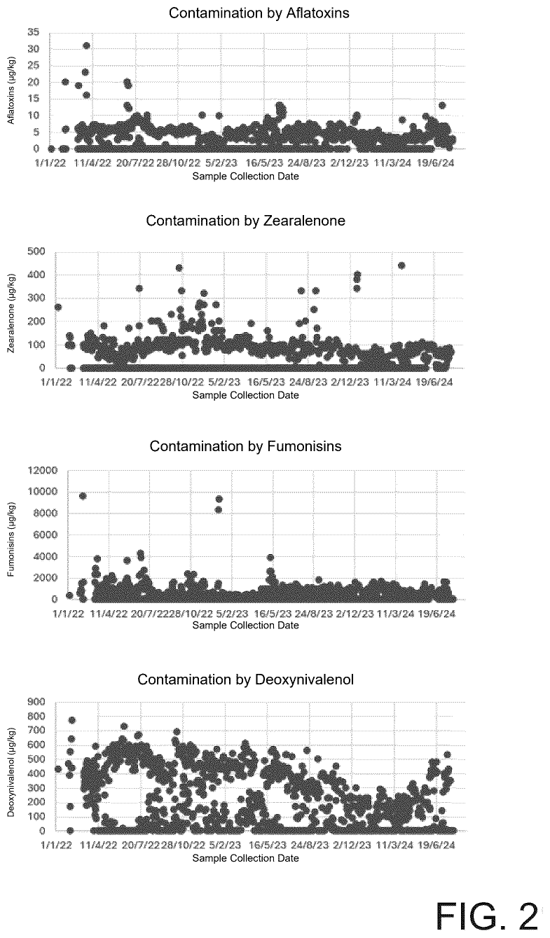

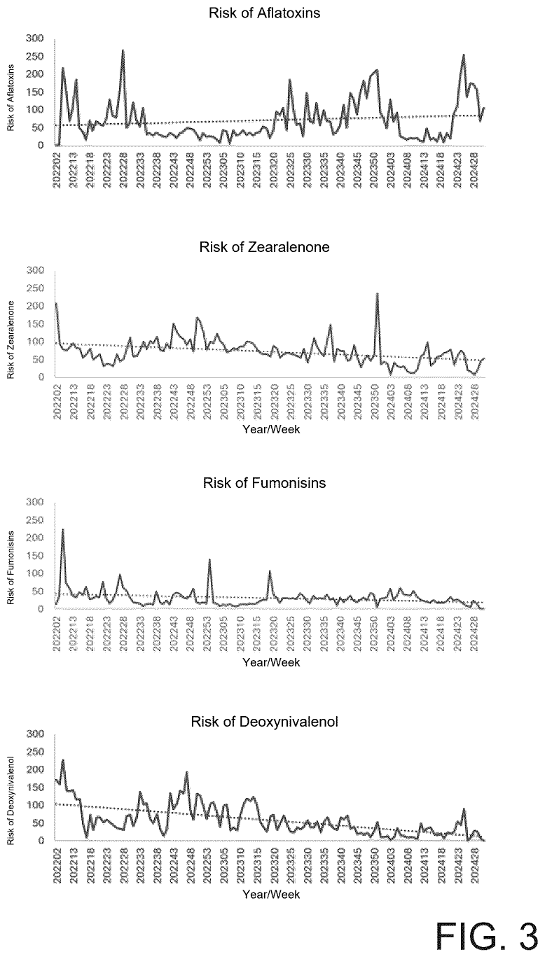

In a Step 6 (6), the calculation of the risk index adjustment is conducted according to the species, age and animal category with specific factor, or FA, this showing value between 10° and 10-10, i.e., from 1 to 0.0000000001, such factor chosen by the user of the method according to his/her needs given the specific livestock situation and, also, in such a way that all the adjusted risk indexes present a standardized numerical base among all the mycotoxins analyzed. Examples of such standardized numerical base can be more clearly seen in FIG. 3 of the patent application, which shows mycotoxin risk index graphs and with all such graphs having a single index ranging from 0 to 300 for several mycotoxins, regardless of the mycotoxin itself. FIG. 2 itself., shows standard verification graphs of mycotoxin contamination, something normal in the state-of-the-art, and, as we can see in the y axis of parts per billion, different mycotoxins may present extremely different concentrations, FIG. 2 showing concentrations ranging from 0 and 35 in case of aflatoxins and from 0 to 12000 in case of zearalenone. Thus, in the data analysis as seen in FIG. 2 graphs, an expert is required to interpret the data obtained on mycotoxin contamination and concentration, given that a concentration in parts per billion can be normal in case of certain mycotoxins and at the same time be extremely harmful in case of other mycotoxins. Thus, the standardized numerical base brought by the use of the specific factor shows a standardized index, as seen in FIG. 3, bringing the livestock producer information much easier to interpret than the mere concentration in parts per billion itself, this not being predicted by the state-of-the-art.

In a main performance, the Risk of Mycotoxins Adjusted for Species, Age and Category, or RMA, is calculated as follows:

$$RMA = RM \times FA$$

Thus, the method shown in this invention patent application discloses an efficient and robust method to create a mycotoxin risk index, applicable to different situations of breeding centers of different livestock producers according to their specific needs, this being something that is not shown in the state-of-the-art.

Such index obtained reflects, thus, the reality of different breeding centers or fields, used to indicate the mycotoxin evolution trend, and the standardized and easy to understand index even when referring to several different mycotoxins, allowing a basis to take decisions, such as, for example, the implementation of specific mitigation means of different mycotoxins.

### BIBLIOGRAPHIC REFERENCES

[1] Mallmann, Adriano Olnei, Alexandro Marchioro, Maurício Schneider Oliveira, Ricardo Hummes Rauber, Paulo Dilkin, e Carlos Augusto Mallmann. 2014. “Comparison of the Efficiency between Two Sampling Plans for Aflatoxins Analysis in Maize”. Brazilian Journal of Microbiology. FapUNIFESP (SciELO). https://doi.org/10.1590/s1517-83822014000100006.
[2] Mallmann, Adriano Olnei, Alexandro Marchioro, Maurício Schneider Oliveira, Luciane Minetto, Liziane Rachel da Silva Wovst, Ricardo Hummes Rauber, Paulo Dilkin, e Carlos Augusto Mallmann. 2013. “Dois Planos de Amostragem Para Análise de Fumonisinas Em Milho”. Ciência Rural. FapUNIFESP (SciELO). https://doi.org/10.1590/s0103-84782013000300029.

1. METHOD TO CALCULATE THE RISK OF MYCOTOXINS IN POULTRY AND SWINE USING CONTAMINATION DATA AND RISK FACTORS AND ADJUSTMENTS,
showing a method which performs a mycotoxin risk index calculation, such method characterized by performing a series of sequential steps, being:
In a Step 1 (1) a period of time for data collection to evaluate the risk of contamination is established, quantity of samples collected in such selected period of time, selection of which mycotoxins will be analyzed, those being chosen according to the needs of the method user;
In a Step 2 (2), the collection of samples of each mycotoxin selected during the period of time chosen for analysis is conducted;
In a Step 3 (3), the average mycotoxin contamination in micrograms per kilogram of each of the collected samples is calculated, and the prevalence as the percentage of positive samples in the selected period of time, such calculations being conducted for each selected mycotoxin;
In a Step 4 (4), the choice for an epidemiological factor, to be applied as a calculation factor in the prevalence calculates in Step 3 (3) is performed and properly chosen by an expert in mycotoxins in production animals according to the needs of a specific livestock situation;
In a Step 5 (5), a calculation of the mycotoxin risk index is performed, such calculation to be applied to each mycotoxin analyzed;
In a Step 6 (6), the calculation of the risk index adjustment is conducted according to the species, age and animal category with specific factor, this showing value between 10° and 10-10, such factor chosen by the user of the method according to his/her specific needs and in such a way that all the adjusted risk indexes present a standardized numerical base among all the mycotoxins analyzed.

2. METHOD TO CALCULATE THE RISK OF MYCOTOXINS IN POULTRY AND SWINE USING CONTAMINATION DATA AND RISK FACTORS AND ADJUSTMENTS, according to claim 1, characterized by, in Step 1 (1), of using a period of collection time chosen from a set of shift, daily, weekly, fortnightly or monthly periods.

3. METHOD TO CALCULATE THE RISK OF MYCOTOXINS IN POULTRY AND SWINE USING CONTAMINATION DATA AND RISK FACTORS AND ADJUSTMENTS, according to claim 1, characterized by, in Step 5 (5), the epidemiological factor showing a value between 1.1 and 1.9, and perform the mycotoxin risk calculation as follows, where FE is the epidemiological factor chosen in Step 4 (4):
$$RM = Contamination \times {Prevalence}^{FE} .$$

4. METHOD TO CALCULATE THE RISK OF MYCOTOXINS IN POULTRY AND SWINE USING CONTAMINATION DATA AND RISK FACTORS AND ADJUSTMENTS, according to claim 1, characterized by, in Step 6 (6), if the risk calculation is performed as follows, where FA is the specific factor chosen in Step 6 (6):
$$RMA = RM \times FA .$$
